(19) United States
(12) Patent Application Publication
(10) Pub. No.: US 2019/0004034 A1
(43) Pub. Date: Jan. 3, 2019

(54) PRODUCING METHOD OF ARTIFICIAL SKIN AND ARTIFICIAL SKIN

(71) Applicant: AMOREPACIFIC CORPORATION, Seoul (KR)

(72) Inventors: Sunghoon LEE, Yongin-si (KR); Il-Hong BAE, Yongin-si (KR); Daejin MIN, Yongin-si (KR); Soojung OH, Yongin-si (KR); Tae Ryong LEE, Yongin-si (KR); Eungyung CHO, Yongin-si (KR)

(73) Assignee: AMOREPACIFIC CORPORATION, Seoul (KR)

(21) Appl. No.: 16/023,045

(22) Filed: Jun. 29, 2018

(30) Foreign Application Priority Data
Jun. 30, 2017 (KR) 10-2017-0083516

(51) Int. Cl. G01N 33/50; C12N 5/071

(52) U.S. Cl. CPC G01N 33/5088; C12N 5/0698; C12N 2502/094; C12N 2533/54; C12N 2502/1323; C12N 2503/06

(57) ABSTRACT

According to an embodiment, a method of producing artificial skin includes mixing fibroblasts and collagen to form a dermis simulating layer, applying keratinocytes on the dermis simulating layer and culturing the same, culturing the dermis simulating layer in a first medium that is a keratinocyte medium, and culturing the dermis simulating layer that is cultured in the first medium in a second medium including a DMEM medium and a F12 medium.

### CROSS-REFERENCE TO RELATED APPLICATION

This application claims priority to and the benefit of Korean Patent Application No. 10-2017-0083516 filed in the Korean Intellectual Property Office on Jun. 30, 2017, the entire contents of which are incorporated herein by reference.

### BACKGROUND OF THE INVENTION

### (a) Field of the Invention

This disclosure relates to a method of producing artificial skin. This disclosure specifically relates to a method of producing artificial skin having skin pigmentation. In addition, this disclosure relates to artificial skin including keratinocytes and a melanocytes.

### (b) Description of the Related Art

Skin is an organ covering the outside of a body, and consists of three layers of an epidermis, a dermis, and a hypodermis from the outmost thereof. The epidermis is mostly composed of keratinocytes of a stratified squamous epithelium. The dermis formed of a matrix protein such as a collagen fiber and an elastic fiber is present beneath the epidermis, and includes blood vessels, nerves, sweat glands, and the like. The hypodermis is formed of adipocytes. The skin maintains its shape through an interaction of the aforementioned various cells and their substances, and performs various functions of adjusting a body temperature, working as a barrier against an external environment, and the like.

The skin may be three-dimensionally restructured as artificial skin by using skin cells and a skin substance such as collagen, elastin, and the like, and the artificial skin is formed of living fibroblasts and keratinocytes and thus shows similar structural and functional characteristics to real skin and accordingly is called reconstructed skin (a skin equivalent). The artificial skin is mainly a polymer composite showing equivalent properties to skin in terms of elasticity, strength, material permeability, and the like, but is different therefrom in terms of not showing life like real skin. The artificial skin may not only be used for replacement (a permanent engrafting type) or reproduction (a temporary covering type), but may also be used in various fields such as skin physiology research, skin stimulus evaluation, skin effect evaluation, and the like.

A pigmentation-induced artificial skin model may be, for example, used for a whitening effect evaluation of cosmetics. Pigmentation of skin is mainly caused by presence of a melanic pigment in the epidermis, and melanin is synthesized by melanocytes, which are dendritic cells in a basal layer of the epidermis. A conventional pigmented artificial skin is formed by using a method of separately culturing keratinocytes and melanocytes, and then co-seeding them to induce development of an epidermis layer.

### SUMMARY OF THE INVENTION

The method of separately culturing keratinocytes and melanocytes and co-seeding them to form an epidermis layer may complicate a process of forming artificial skin. In addition, since conditions for substantially culturing keratinocytes and melanocytes together are difficult to set, and thus the melanocytes are not wholly present in a final artificial skin model, the artificial skin model may not function as a normal pigmented artificial skin model.

Accordingly, a production method of artificial skin according to an embodiment provides a more similar pigmented artificial skin to real pigmented skin by using keratinocytes.

According to an embodiment, a method of producing artificial skin includes mixing fibroblasts and collagen to form a dermis simulating layer, applying keratinocytes on the dermis simulating layer and culturing the same, culturing the dermis simulating layer in a first medium that is a keratinocyte medium, and culturing the dermis simulating layer that is cultured in the first medium in a second medium including a DMEM medium and an F12 medium.

The keratinocytes may include melanocyte stem cells.

The second medium may contain a component inducing differentiation of the melanocyte stem cells.

The first medium may be disposed to contact a bottom surface of the dermis simulating layer, and an opposite surface to the bottom surface where the first medium is disposed may be exposed to the air.

The culturing in the second medium may proceed directly after the culturing in the first medium.

The culturing in the first and second media may be performed within 20 days in total.

The culturing period in the first medium may be equal to or longer than the culturing period in the second medium.

The second medium may be prepared by mixing the DMEM medium and the F12 medium in a ratio of about 1:1 to about 4:1.

The second medium may further include at least one selected from hydrocortisone, triiodothyronine, cholera toxin, ascorbic acid, and calcium chloride (CaCl₂).

The culturing in the second medium may proceed in an exposure state to the air.

Another embodiment provides artificial skin including a dermis simulating layer and an epidermis simulating layer on the dermis simulating layer, wherein the epidermis simulating layer includes an epidermis basal layer part being adjacent to the dermis simulating layer and a horny layer part exposed to the air, and keratinocytes present in the horny layer and melanocytes present in the epidermis basal layer originate from the same person.

Still another embodiment provides a method of evaluating an effect of a pigmentation controlling agent, which includes applying the pigmentation controlling agent to artificial skin formed according to the aforementioned production method or artificial skin having the aforementioned structure and comparing a pigmentation degree of the artificial skin to which the pigmentation controlling agent is applied with a pigmentation degree of artificial skin to which the pigmentation controlling agent is not applied.

The pigmentation controlling agent may be applied through direct contact with the artificial skin.

The production method according to an embodiment may induce differentiation of a melanocyte stem cell present among keratinocytes and thus provide pigmented artificial skin without using the melanocyte. Artificial skin according to the production method is similar to real skin and thus may provide improved reliability of evaluation data when used as a skin whitening evaluation model.

### BRIEF DESCRIPTION OF THE DRAWINGS

FIG. 1 is a flowchart illustrating a production method of artificial skin according to an embodiment,

FIG. 2 is a microscope photograph showing a cross-section of artificial skin according to Example 1 after dying H&E tissues, and

FIG. 3 is a microscope photograph showing a cross-section of artificial skin according to Comparative Example 1 after dying H&E tissues.

### DETAILED DESCRIPTION OF THE EMBODIMENTS

Hereinafter, exemplary embodiments of the present disclosure will be described in detail, and may be easily performed by a person having ordinary skill in the related art. However, this disclosure may be embodied in many different forms, and is not to be construed as limited to the exemplary embodiments set forth herein.

In the present specification, “artificial skin” is skin that is three-dimensionally reconstructed by using skin cells and a skin substance such as collagen and the like, and includes any polymer composite showing similar structural and functional characteristics to those of real skin.

In the present specification It will be understood that when an element such as a layer, film, region, or substrate is referred to as being “on” another element, it may be directly on the other element or intervening elements may also be present. In contrast, when an element is referred to as being “directly on” another element, there are no intervening elements present.

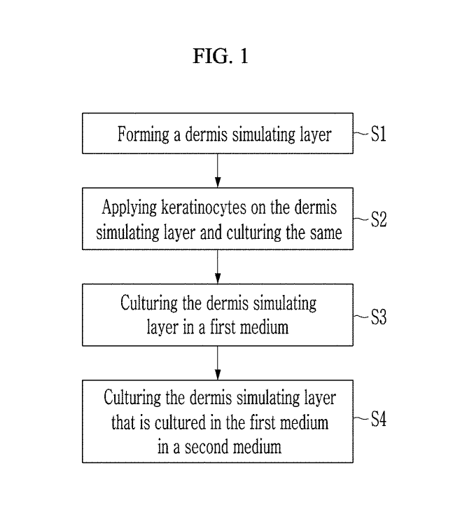

Hereinafter, the production method of artificial skin according to an embodiment is illustrated with reference to FIG. 1.

FIG. 1 is a flowchart illustrating the production method of artificial skin according to an embodiment.

Referring to FIG. 1, a method of producing artificial skin according to an embodiment includes forming a dermis simulating layer (S1), applying keratinocytes on the dermis simulating layer and culturing the same (S2), culturing the dermis simulating layer in the first medium (S3), and culturing the dermis simulating layer that is cultured in the first medium in a second medium (S4).

First, the formation of a dermis simulating layer (S1) is illustrated.

A dermis is a skin layer beneath an epidermis formed of connective tissues, and thus works as a buffer and protects a body from pressure as well as stresses and strains. The dermis is firmly connected with the epidermis through a basement membrane. The dermis provides a substrate for supporting various components such as blood vessels, nerves, and the like that are inherent in the structure.

The dermis simulating layer is formed by using a mixed material of fibroblasts and collagen in with a ratio that is correlated with that of real skin. The dermis simulating layer may consist of at least two layers, for example, a first dermis layer containing the collagen and a second dermis layer containing the fibroblasts. In this case, a dermis contraction phenomenon of the artificial skin may be further alleviated.

The first dermis layer may contain collagen as well as an extracellular matrix such as elastin, chitosan, glycosaminoglycans (GAGs), and hyaluronic acid (HA).

The second dermis layer may be present on the first dermis layer, and contains fibroblasts as well as an extracellular matrix such as elastin, chitosan, glycosaminoglycans (GAGs), and hyaluronic acid (HA).

The collagen may originate from natural collagen such as that from a cow, a rat tail, or a fish, or may be produced through genetic engineering, and may contract under presence of fibroblasts.

The dermis simulating layer may generally have a thickness of greater than or equal to about 0.05 cm, and particularly, in a range from about 0.05 to about 2 cm, but the thickness may be increased or decreased, as long as it produces no damage to advantageous characteristics of the artificial skin according to the present invention.

As aforementioned, the dermis simulating layer may be, for example, cultured for about 5 days to about 9 days, about 6 days to about 8 days, or about 7 days, after being formed by the mixed material of fibroblasts and collagen.

When the dermis simulating layer is formed, keratinocytes are applied thereon, and the dermis simulating layer is cultured (S2).

The keratinocytes constitute about 80% to about 90% of cells of the epidermis layer, are formed through mitosis in the basal layer, which is the deepest layer of the epidermis layer, and then rise toward a skin surface.

The culturing after applying the keratinocytes (S2) is a first step for forming an epidermis simulating layer of the artificial skin, wherein the keratinocytes may, for example, originate from a human. The keratinocytes may be any commercially available keratinocytes, which may not only be directly separated from a human or cultured after separation, but may also be induced from a different cell from a human cell. The commercially available human keratinocytes may include NHEK-Neo, Pooled (Neonatal Normal Human Epidermal Keratinocytes, Pooled: Product No. 00192906, Tissue registration No. P867, white people), NHEK-Neo (Product No. 00192907, Tissue registration No. 20647, white people), NHEK-Adult (Product No. 00192627, Tissue registration No. 21155, white people), NHEK-Neo (Product No. 00192907, Tissue registration No. 18080, black people), which are provided by Lonza. For example, the commercially available human keratinocytes may include HEKn (Human Epidermal Keratinocytes, neonatal: Product No. C0015C, Tissue registration No. 1781129, white people), and HEKn (Product No. C0015C, Tissue registration No. 1803827, black people), which are provided by Thermo Fisher Scientific Company. In order to maintain a keratinocyte's nature of growing while adhered to a basement membrane, a dish may be coated with about 0.1 to 0.2% gelatin or about 0.1 to 0.2 mg/ml collagen type IV. The cells may be cultured in an incubator at about 35 to 37° C. under about 5 to 10% CO₂ and sub-cultured when density thereof reaches about 70 to 80%.

The keratinocytes may be, for example, applied on the dermis simulating layer through seeding, and the culture may, for example, proceed for about 1 day to about 4 days, about 1 day to about 3 days, or about 1 day to about 2 days, but is not limited thereto.

Among the keratinocytes, melanocyte stem cells may be included in a small amount. The melanocyte stem cells are differentiated into melanocytes through culturing in first and second media, which will be described later.

After the application and culturing of keratinocytes (S2), the cells are cultured in the first medium, which is a keratinocyte medium (S3).

Herein, the “keratinocyte medium” denotes a medium for culturing human keratinocytes, and can be any medium known in the related art. For example, the keratinocyte medium may include a bovine pituitary extract (BPE), a human epidermal growth factor (hEGF), bovine insulin, hydrocortisone, gentamicin, and amphotericin-B (GA-1000). In addition, epinephrine and transferrin may be further added to the components. The medium may be a commercially available one, for example, CnT-3D-PR (CellnTEC Advanced Cell Systems AG).

The first medium may be disposed to contact a bottom surface of the dermis simulating layer, and an opposite surface to the bottom surface of the first medium is disposed may be exposed to the air. The culture in the first medium may, for example, proceed for about 3 days to about 10 days, about 4 days to about 10 days, about 5 days to about 9 days, or about 4 days to about 8 days, but is not limited thereto.

Subsequently, the dermis simulating layer cultured in the first medium is cultured in a second medium including a DMEM medium and an F12 medium (S4).

This mixed medium may be prepared by mixing commercially available DMEM medium and F12 medium, for example, in a ratio of about 1:1 to about 4:1, and specifically about 2:1 to about 3:1, but is not limited thereto. The DMEM (Dulbecco's modified Eagle medium) is a medium for culturing a generally-used animal cell, and may include amino acids, vitamins, inorganic salts, glucoes, lipids, indicators, serums, and the like.

The second medium may include 100% of the DMEM medium and the F12 medium based on a total amount of a culture medium, but additionally, it may include at least one selected from hydrocortisone, triiodothyronine, cholera toxin, ascorbic acid, and calcium chloride (CaCl₂).

The culturing in the second medium may proceed in an exposure state to the air like the culturing in the first medium.

The culturing in the second medium may subsequently proceed, for example, for about 4 days to about 15 days, about 5 days to about 13 days, about 6 days to about 10 days, or about 7 days to about 9 days, directly after the culturing in the first medium, but is not limited thereto. However, the culturing periods in the first and second media may be adjusted, for example, within about 20 days. For example, the culturing period in the first medium may be equal to or longer than the culturing period in the second medium.

After S2 to S4, an artificial skin structure having the epidermis simulating layer on the dermis simulating layer is obtained.

The production method of artificial skin according to an embodiment may differentiate melanocyte stem cells present among keratinocytes seeded on the dermis simulating layer before differentiating melanocytes through the sequential culturing in the first medium (S3) and the culturing in the second medium (S4).

When artificial skin is formed according to the aforementioned method, melanocytes are uniformly distributed on the epidermis basal layer, their distribution density is substantially consistent on a parallel surface to the surface of a dermis replacement, and thus an artificial skin model that is substantially similar to real human skin may be obtained.

In other words, an artificial skin model wherein keratinocytes and melanocytes are effectively present may be realized by inducing differentiation of melanocyte stem cells present among keratinocytes without separately culturing the melanocytes.

Another embodiment of the present invention provides artificial skin including a dermis simulating layer and an epidermis simulating layer on the dermis simulating layer, wherein the epidermis simulating layer includes an epidermis basal layer part being adjacent to the dermis simulating layer and a horny layer part exposed to the air, and keratinocytes present in the horny layer and melanocytes present in the epidermis basal layer originate from the same person.

The origination of the keratinocytes and the melanocytes of the artificial skin from the same person means that the melanocytes are not cultured separately from the keratinocytes but are differentiated from melanocyte stem cells present among keratinocytes during the production process of the artificial skin.

Yet another embodiment of the present invention provides a method of evaluating an effect of a pigmentation controlling agent including applying the pigmentation controlling agent to the artificial skin or artificial skin obtained according to the aforementioned production method, and then comparing a pigmentation degree of the artificial skin to which the pigmentation controlling agent is applied with a pigmentation degree of artificial skin to which the pigmentation controlling agent is not applied.

The term “pigmentation controlling” indicates changing, for example, directly or indirectly increasing or decreasing or suppressing a compositional or inductive pigmentation level of skin. The pigmentation controlling agent may be a pigmentation-increasing or decreasing agent.

The pigmentation controlling agent may be applied to the artificial skin through direct contact. The pigmentation controlling agent may be, for example, whitening cosmetics, but is not limited thereto.

Hereinafter, the present disclosure is illustrated in more detail with reference to examples. However, these examples are exemplary, and the present disclosure is not limited thereto.

### Production of Artificial Skin

### Example 1

### (1) Step 1: Production of Dermis Simulating Layer

Fibroblasts were mixed with collagen to form a dermis simulating layer, and then the dermis simulating layer was cultured for one week.

### (2) Step 2: Keratinocyte Application and Culture

After the 1^{st} step, keratinocytes (HEKn, Thermo Fisher Scientific Company) were applied on the dermis simulating layer and then cultured for 2 days.

### (3) Step 3: Culture in First Medium

After the 2^{nd} step, air culturing proceeded for 8 days by disposing a first medium (CnT-3D-PR, CellnTEC) to contact a bottom part of the reconstructed dermis, while an epidermis layer was exposed to the air.

### (4) Step 4: Culture in Second Medium

After the 3^{rd} step, the air culturing additionally proceeded for 6 days in a second medium to obtain an artificial skin model. The second medium was prepared by mixing a DMED medium (LM001-05, Welgene Inc.) and an F12 medium (LM010-01, Welgene Inc.) in a ratio of 2.5:1 and adding hydrocortisone, triiodothyronine, cholera toxin, ascorbic acid, and calcium chloride (CaCl₂) thereto.

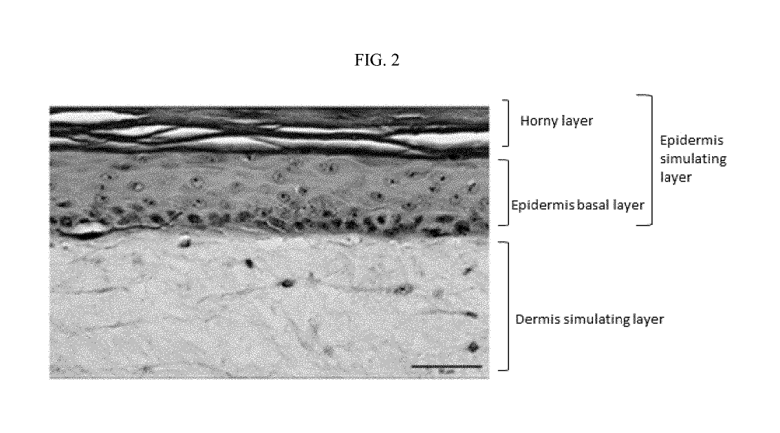

FIG. 2 is a microscope photograph showing a cross-sectional view of the artificial skin model.

### Comparative Example 1

Artificial skin was prepared according to the same method as Example 1, except for not performing the culturing in the second medium (the 4^{th} step).

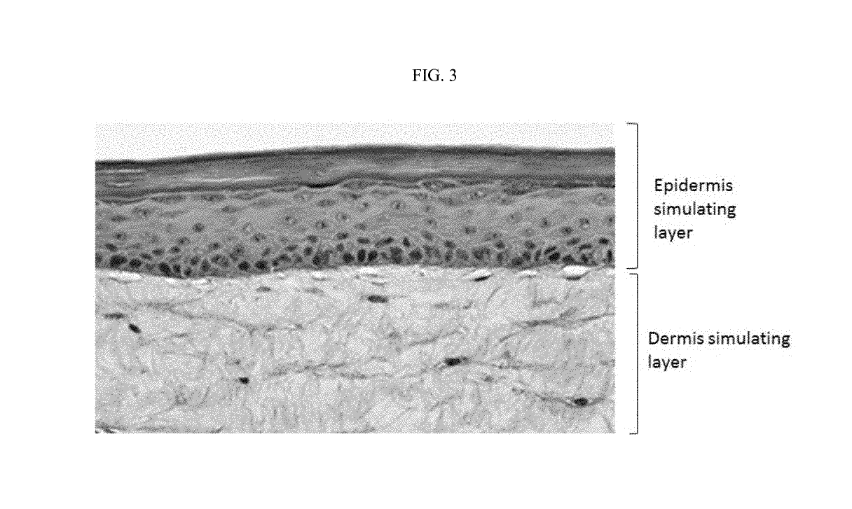

FIG. 3 is a microscope photograph showing a cross-sectional view of the artificial skin model.

### Evaluation: Cross Section Examination of Artificial Skin Model

The cross-sections of the artificial skin models according to Example 1 and Comparative Example 1 were examined with a microscope.

FIGS. 2 and 3 are microscope photographs showing the cross-sections of the artificial skins according to Example 1 and Comparative Example 1 after dying an H&E tissue.

Referring to FIGS. 2 and 3, a pigmentated artificial skin was obtained by using a keratinocyte alone and adjusting a medium for culturing artificial skin. In detail, the same keratinocytes were used, but melanocytes differentiated from melanocyte stem cells in the base of the epidermis layer were found in Example 1 of the present invention (FIG. 2), while melanocytes were not found in the base of the epidermis layer in Comparative Example 1 (FIG. 3).

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

1. A method of producing artificial skin of a multilayered stratified structure comprising a dermis simulating layer and an epidermis simulating layer, comprising:
(a) mixing fibroblasts and collagen to form a dermis simulating layer;
(b) applying keratinocytes on the dermis simulating layer and culturing the resulting dermis simulating layer applied with keratinocytes to form a multilayer structure of keratinocytes-containing epidermis simulating layer and dermis simulating layer;
(c) culturing the multilayer structure of keratinocytes-containing epidermis simulating layer and dermis simulating layer obtained in (b) in a first medium suitable for culturing human keratinocytes to give a multilayered stratified structure of epidermis simulating layer and dermis simulating layer; and
(d) culturing the multilayered stratified structure of epidermis layer and dermis simulating layer obtained in (c) in a second medium including a DMEM medium and an F12 medium.

2. The method of claim 1, wherein the keratinocytes of (b) include melanocyte stem cells.

3. The method of claim 2, wherein the second medium of (d) includes a component inducing differentiation of the melanocyte stem cells.

4. The method of claim 1, wherein the (c) further comprises air-lifting culturing of the multilayer structure of keratinocytes-containing epidermis simulating layer and dermis simulating layer wherein a bottom surface of the dermis simulating layer is in contact with the first medium and the keratinocytes-containing epidermis simulating layer is exposed to the air.

5. The method of claim 1, wherein the (d) of culturing in the second medium proceeds directly after the culturing of (c).

6. The method of claim 1, wherein the (c) and (d) are carried out for about 20 days in total.

7. The method of claim 6, wherein a duration of the culturing of (c) is equal to or longer than a duration of the culturing of (d).

8. The method of claim 1, wherein the second medium of (d) comprises the DMEM medium and the F12 medium in a ratio ranging from about 1:1 to about 4:1.

9. The method of claim 1, wherein the second medium of (d) further comprises at least one selected from hydrocortisone, triiodothyronine, cholera toxin, ascorbic acid, and calcium chloride (CaCl₂).

10. The method of claim 1, wherein the culturing of (d) is carried out while the structure is exposed to air.

11. Artificial skin comprising a dermis simulating layer and a stratified epidermis simulating layer on the dermis simulating layer,
wherein the stratified epidermis simulating layer comprises an epidermis basal sublayer being adjacent to the dermis simulating layer and an uneven surface sublayer exposed to the air;
keratinocytes are present in the uneven surface sublayer and melanocytes are present in the epidermis basal sublayer; and
wherein the keratinocytes and melanocytes are originated from a same person.

12. A method for evaluating an effect of a pigmentation controlling agent on skin, comprising:
applying the pigmentation controlling agent to an artificial skin produced according to claim 1; and
comparing a pigmentation degree of the artificial skin to which the pigmentation controlling agent is applied with a pigmentation degree of the artificial skin to which the pigmentation controlling agent is not applied, under the same condition.

13. The method of claim 12, wherein the pigmentation controlling agent is applied through direct contact with the artificial skin.
